(19) United States
(12) Patent Application Publication
(10) Pub. No.: US 2023/0148841 A1
(43) Pub. Date: May 18, 2023

(54) ENDOSCOPE INTERNAL DYNAMIC SEAL

(71) Applicant: Cook Medical Technologies LLC, Bloomington, IN (US)

(72) Inventors: Michael J. Brecht, Pfafftown, NC (US); Christopher A. Carruthers, Winston-Salem, NC (US); Marc Spencer, Winston-Salem, NC (US)

(21) Appl. No.: 17/975,089

(22) Filed: Oct. 27, 2022

Related U.S. Application Data
(60) Provisional application No. 63/280,700, filed on Nov. 18, 2021.

(51) Int. Cl. A61B 1/00; A61B 1/018

(52) U.S. Cl. CPC A61B 1/00137; A61B 1/018; A61B 1/0057

(57) ABSTRACT

Disclosed herein is a medical device including a dynamic seal and a movable member. The device allows the movable member to translate proximally and distally through the dynamic seal while fluid does not flow proximally to the dynamic seal member.

### CROSS-REFERENCE TO RELATED APPLICATIONS

This application claims the benefit of U.S. Patent Application Ser. No. 63/280,700, filed Nov. 18, 2021, the entirety of which is incorporated herein by reference for all purposes.

### FIELD

The present disclosure relates to medical devices. More particularly, the disclosure relates to internal seals within endoscope systems.

### BACKGROUND

The statements in this section merely provide background information related to the present disclosure and may not constitute prior art.

Internal body cavities and body lumens may become blocked, or the walls surrounding them may develop growths. In some cases, removal of these blockages or growths, or other treatment thereof, may be necessary. Endoscopic or other minimally invasive techniques may be used to treat these situations.

One type of treatment includes the use of catheters or other endoscopic devices that are inserted into the body lumen or cavity and toward the area where treatment is desired. Insertion of the endoscope to the target area can allow for visualization of the target area and a determination of the desired procedure and the specific location of the area to be treated.

In general, endoscopes have been designed to be operated with the same fundamental mechanisms, and have not had transformational improvements. Endoscopes generally include a camera and a set of wheels that an operator, such as a physician, operates with a first hand (in some cases, the left hand) to control scope deflection, while the second (generally, right) hand switches between the insertion tube of the endoscope and the accessory channel in order to control scope and device advancement, respectively, through the anatomy of a patient.

Specialized endoscopes, tailored to specific procedures, are becoming more common in the field of endoscopy with the trend from reusable scopes to disposable scopes. Whereas traditional endoscopes include accessory channel(s) that are fixed from axial movement, specialized endoscopes may include a feature in which the accessory channels may move proximally or distally so as to change the camera between forward-viewing and side-viewing configurations. Escape of gas from the proximal end of the insertion tube should be minimized, and the escape of gas has traditionally been minimized by use of static seals. Given the proximal or distal movement of accessory channels in some specialized endoscopes, static sealing methods used in traditional endoscopes are not possible between the accessory channels and the insertion tube. Further, in such specialized endoscopes, deflection wires, coils, or cables used to provide deflection of the distal end of the endoscope must be exposed distally to allow the accessory channels to deflect between the bending section linkages. Accordingly, static sealing around the deflection wires is insufficient. Additionally, in such specialized endoscopes, electrical or fiber optic wires are used to provide power, one-way signaling, two-way signaling, communication for data, or light at the distal end of the endoscope to power LED(s), other sensors, circuits, or light, and static sealing around electrical or fiber optic wires is insufficient.

### SUMMARY

In an example, the present disclosure provides a medical device. The medical device includes an elongate tube including a lumen extending therethrough, the elongate tube defining a longitudinal axis therethrough. The medical device further includes a movable member extending longitudinally at least partially within the lumen. The medical device further includes a dynamic seal, including a dynamic seal member at a proximal end of the elongate tube, the movable member configured to translate proximally and distally through the dynamic seal member, and a flexible seal through which the movable member extends longitudinally. The dynamic seal member is configured to prevent fluid from flowing proximally to the dynamic seal member. The flexible seal member may be an elastomeric seal. The flexible seal may include an amorphous sealing material. The movable member may be an accessory channel of an endoscope system. The movable member may be a deflection wire of an endoscope system. The movable member may be an electrical wire or a fiber-optic wire of an endoscope system. The medical device may include a plurality of movable members and the dynamic seal may include a plurality of flexible seals, one movable member of the plurality of movable members each extending extend through one flexible seal of the plurality of flexible seals. The medical device may include a second dynamic seal member wherein the dynamic seal member may be within the second dynamic seal member and the second dynamic seal member may be rotationally decoupled from the dynamic seal member. The dynamic seal member may be variably adjustable and configured to increase or decrease friction on the movable member.

In another example, the present disclosure provides a medical device. The medical device includes an elongate tube including a lumen extending therethrough, the elongate tube defining a longitudinal axis therethrough. The medical device further includes a movable member extending longitudinally at least partially within the lumen. The medical device further includes a first dynamic seal member at a proximal end of the elongate tube, the movable member configured to translate proximally and distally through the first dynamic seal member. The medical device further includes a flexible seal through which the movable member extends longitudinally. The medical device further includes a second dynamic seal member rotationally decoupled from the first dynamic seal member. The first dynamic seal member is within the second dynamic seal member. The first dynamic seal member is configured to prevent fluid from flowing proximally to the first dynamic seal member. The flexible seal may be an elastomeric seal. The flexible seal may include an amorphous sealing material. The movable member may be an accessory channel of an endoscope system. The movable member may be a deflection wire of an endoscope system. The movable member may be an electrical wire or a fiber-optic wire of an endoscope system. The medical device may include a plurality of movable members and the first dynamic seal member may include a plurality of flexible seals, one movable member of the plurality of movable members each extending through one flexible seal of the plurality of flexible seals. The medical device may include a catch proximal to the second dynamic seal member, the second dynamic seal member configured to move between a locked configuration and an unlocked configuration, the second dynamic seal member being biased to the locked configuration. When the second dynamic seal member is in the unlocked configuration, the second dynamic seal member may be rotatable about the longitudinal axis relative to the first dynamic seal member. When the second dynamic seal member is in the locked configuration, the second dynamic seal member may engage with the catch and not be rotatable about the longitudinal axis relative to the first dynamic seal member.

In yet another example, the present disclosure provides a medical device. The medical device includes a lumen extending therethrough, the elongate tube defining a longitudinal axis therethrough. The medical device further includes a movable member extending longitudinally at least partially within the lumen. The medical device further includes a dynamic seal includes a dynamic seal member at a distal end of the elongate tube, the movable member configured to translate proximally and distally through the dynamic seal member and a flexible seal through which the movable member extends longitudinally. The dynamic seal member may be configured to prevent fluid from flowing proximally to the dynamic seal member.

Further areas of applicability will become apparent from the description provided herein. It should be understood that the description and specific examples are intended for purposes of illustration only and are not intended to limit the scope of the present disclosure.

### DRAWINGS

In order that the present disclosure may be well understood, there will not be described various forms thereof, given by way of example, reference being made to the accompanying drawings. The components in the figures are not necessarily to scale. Moreover, in the figures, like-referenced numerals designate corresponding parts through the different views.

FIG. 1 illustrates a perspective view of an example of an endoscope system having a handle constructed according to the principles of the present disclosure;

FIG. 2 illustrates a perspective view of an example of the elements of a dynamic seal, together with movable member(s) and elongate tube, in accordance with the principles of the present disclosure;

FIG. 3 illustrates a perspective view of an example of a dynamic seal as assembled, in accordance with the principles of the present disclosure;

FIG. 4 illustrates a diametrical longitudinal cross-sectional view of an example of a dynamic seal as assembled, in accordance with the principles of the present disclosure;

FIG. 5 illustrates a chordal longitudinal cross-sectional view of an example of a dynamic seal as assembled, in accordance with the principles of the present disclosure;

FIG. 6 illustrates a top view of an example of a middle dynamic seal member in accordance with the principles of the present disclosure;

FIG. 7 illustrates a perspective view of an example of proximal and distal middle dynamic seal members and seals in accordance with the principles of the present disclosure;

FIG. 8 illustrates the example of the elements of the dynamic seal of FIG. 2 including deflection wires, in accordance with the principles of the present disclosure;

FIG. 9 illustrates a perspective view of an example of a handle including an example of a dynamic seal with a lower dynamic seal member shown as see-through to show a spring internal to the lower member, in accordance with the principles of the present disclosure; and

FIG. 10 illustrates a perspective view of the example of the handle of FIG. 9 with the lower dynamic seal member shown as see-through, the lower dynamic seal member moved distally away from the upper dynamic seal member to compress the spring internal to the lower dynamic seal member, in accordance with the principles of the present disclosure.

The drawings described herein are for illustration purposes only and are not intended to limit the scope of the present disclosure in any way.

### DETAILED DESCRIPTION

The following description is merely exemplary in nature and is not intended to limit the present disclosure, application, or uses. It should be understood that throughout the drawings, corresponding reference numerals indicate like or corresponding parts and features.

In adding reference denotations to elements of each drawing, although the same elements are displayed on a different drawing, it should be noted that the same elements have the same denotations. In addition, in describing one aspect of the present disclosure, if it is determined that a detailed description of related well-known configurations or functions blurs the gist of one aspect of the present disclosure, it will be omitted.

In the following discussion, the terms “proximal” and “distal” will be used to describe the opposing axial ends of the device, as well as the axial ends of various component features. The term “proximal” is used in its conventional sense to refer to the end of the device (or component) that is closest to the medical professional during use of the assembly. The term “distal” is used in its conventional sense to refer to the end of the device (or component) that is initially inserted into the patient, or that is closest to the patient during use. The term “longitudinal” will be used to refer to an axis that aligns with the proximal-distal axis of the device (or component). The terms “radially” and “radial” will be used to refer to elements, surfaces, or assemblies relative to one another that may extend perpendicularly from a longitudinal axis. The terms “circumference,” “circumferentially,” and “circumferential” will be used to refer to elements, surfaces, or assemblies relative to one another encircling a longitudinal axis at a radius. The terms “diameter” and “diametrical” refer to a line passing through a circle that touches two points on the circumference of the circle and the center of the circle. The terms “chord” and “chordal” refer to a line passing through a circle that touches two points on the circumference of the circle but does not touch the center of the circle.

The uses of the terms “a” and “an” and “the” and similar referents in the context of describing the present disclosure (especially in the context of the following claims) are to be construed to cover both the singular and the plural, unless otherwise indicated herein or clearly contradicted by context. The use of the term “plurality of” is defined by the Applicant in the broadest sense, superseding any other implied definitions or limitations hereinbefore or hereinafter unless expressly asserted by the Applicant to the contrary, to mean a quantity of more than one. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context.

As used herein the terms “comprise(s),” “include(s),” “having,” “has,” “can,” “contain(s),” and variants thereof, are intended to be open-ended transitional phrases, terms, or words that do not preclude the possibility of additional acts or structures. The present description also contemplates other examples “comprising,” “consisting of,” “and consisting essentially of,” the examples or elements presented herein, whether explicitly set forth or not.

In describing elements of the present disclosure, the terms 1^{st}, 2^{nd}, first, second, A, B, (a), (b), and the like may be used herein. These terms are only used to distinguish one element from another element, but do not limit the corresponding elements irrespective of the nature or order of the corresponding elements.

Unless otherwise defined, all terms herein, including technical or scientific terms, have the same meanings as those generally understood by those skilled in the art to which the present disclosure pertains. Such terms as those defined in a generally used dictionary are to be interpreted as having meanings equal to the contextual meanings in the relevant field of art.

As used herein, the term “about,” when used in the context of a numerical value or range set forth means a variation of ±15%, or less, of the numerical value. For example, a value differing by ±15%, ±14%, ±10%, or ±5%, among others, would satisfy the definition of “about,” unless more narrowly defined in particular instances.

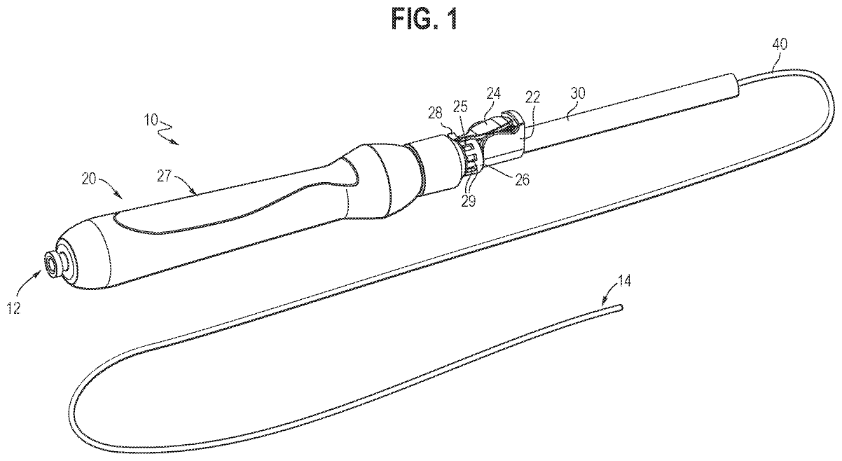

Referring to FIG. 1, an example of an endoscope system 10 is illustrated. The endoscope system 10 extends from proximal end 12 to distal end 14, and includes a handle 20. The endoscope system 10 also includes an elongate tube 30 which is engaged with the handle 20 such that it can rotate relative to the handle 20. The elongate tube 30 may be a flexible tube with at least one lumen 38 running throughout its length. In one aspect, the elongate tube 30 may be made of a braided material, such as a polyether block amide (including, for example, PEBAX) with a polytetrafluoroethylene (“PTFE”) liner to provide sufficient torqueability and pushability. Other potential materials for the elongate tube 30 include, but are not limited to, polyethylene, polypropylene, and nylon.

The endoscope system may further include a movable member 40 running through the elongate tube 30 and/or the handle 20. Movable member 40 may be designed as individual elongated tubes that may be movable within the lumen 38 of the elongate tube 30, thus allowing longitudinal movement of the movable member 40 with respect to the elongate tube 30. Examples of movable member 40 may include accessory channel(s), deflection wire(s), electrical wire(s), and fiber-optic wire(s). Electrical wire(s) or fiber-optic wires may provide power, one-way signaling, two-way signaling, communication for data, or light at the distal end of the endoscope to power light emitting diode(s) (LED(s)), other sensors, circuits, or light. When the elongate tube 30 is in a straight configuration, elongate tube 30 defines a longitudinal axis running centrally therethrough, which represents the axis about which elongate tube 30 may rotate relative to handle 20. In practice, even when elongate tube 30 is not in a straight configuration, the longitudinal axis is still defined at the same position, and the portion of elongate tube 30 that is outside of the body of the patient and that substantially surrounds the longitudinal axis will be rotatable about said axis.

While FIG. 1 illustrates single movable member 40, an endoscope system 10 may include two movable members 40, three movable members 40, or more. For example, a single, larger movable member 40 may be used to accommodate larger endoscopic tools. Further, in lieu of individual movable member(s) 40, a single elongate tube 30 may be used with two or more channel lumens running through it. The movable member(s) 40 may individually range in diameter anywhere from 0.5 millimeter to 20 millimeters, or from 1 millimeter to 10 millimeters. The movable member(s) 40 may extend from proximal of or past handle 20, through lumen 38, and through distal end 14. Movable member(s) 40 may have an open end on both ends, and various tools, devices, and cameras may be inserted into and removed from movable member(s) 40. While movable member(s) 40 have been illustrated to extend well beyond the end of elongate tube 30 in FIG. 1, this drawing is not necessarily to scale, and elongate tube 30 may have a greater length.

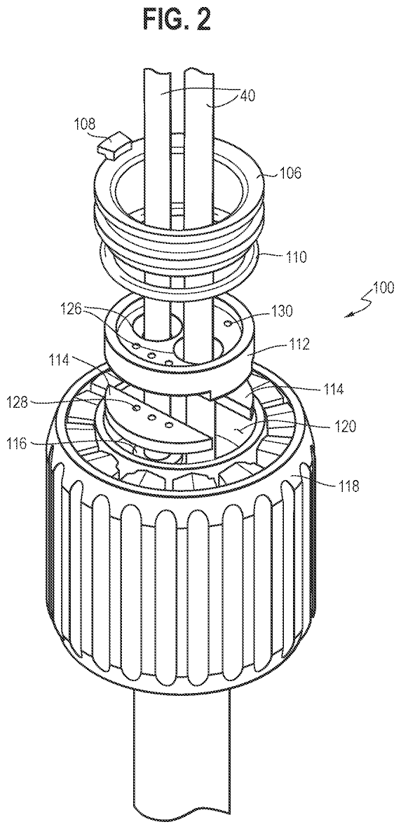

FIG. 2 illustrates a perspective view of an example of the elements of a dynamic seal 100, together with movable member(s) 40 in the form of two accessory channels, and elongate tube 30. Movable member(s) 40 such as accessory channel(s) may move proximally or distally to change endoscope system 10 between forward-viewing and side viewing configurations, which requires a dynamic sealing between movable member(s) 40 and elongate tube 30. Additionally, dynamic sealing is required around deflection wires 402, 404 used to provide deflection of distal end 14 of endoscope system 10.

As illustrated by FIG. 2, sealing between the inner lumen of elongate tube 30 and movable member(s) 40 such as accessory channel(s) or deflection wires 402, 404 may be affected by attachment of lower dynamic seal member 118 around the proximal end of elongate tube 30. Alternatively, though not shown in the example illustrated in FIG. 2, instead of at the proximal end of elongate tube 30, one or more dynamic seal members may be positioned at the distal end of elongate tube 30 and configured to prevent pressurized fluid from flowing proximally to the dynamic seal while allowing movable member(s) to translate through the dynamic seal in distal and proximal directions. Moveable member(s) 40 are then passed through seal 110, which is constrained by upper dynamic seal member 106, upper dynamic seal member 106 proximal to lower dynamic seal member 118. Movable member(s) 40 also pass through upper dynamic seal member 106. Upper dynamic seal member 106 may include stop 108 to preclude complete 360-degree rotation about the longitudinal axis by stop 108 confronting, for example, a catch. Middle dynamic seal member 112 may be a seat for seals 110 and 116 and arcuate seals 114 but may be free floating between upper dynamic seal member 106 and lower dynamic seal member 118 such that middle dynamic seal member 112 may freely rotate. Upper dynamic seal member 106 and lower dynamic seal member 118 may rotate about the longitudinal axis, relative to and around middle dynamic seal member 112, such that middle dynamic seal member 118, seals 110 and 116 and arcuate seals 114, movable member(s) 40, and deflection wires are decoupled from the rotation and remain fixed in position while maintaining sealing. During rotation of upper dynamic seal member 106 and lower dynamic seal member 118, inner surface 120 of lower dynamic seal member 118 may confront middle dynamic seal member 112. FIG. 2 illustrates a plurality of seals, including seals 110 and 116 and arcuate seals 114. Though not shown, in an example of a dynamic seal, a seal may be in the form of a single layer with a specific opening geometry for each of upper dynamic seal member 106, middle dynamic seal member 112, and lower dynamic seal member 118. Alternatively, though not shown, in an example of a dynamic seal, a seal may be in the form such that moveable member(s) 40 of varying diameter and/or geometrical shape may pass through the seal. As illustrated in FIG. 2, dynamic seal 100 is positioned at the proximal end of elongate tube 30, but the dynamic seal of the present invention may be placed anywhere along the length of elongate tube 30. The dynamic seals of the present invention are configured to prevent pressurized fluid from flowing proximally to the dynamic seal while allowing movable member(s) to translate through the dynamic seal in distal and proximal directions.

Middle dynamic seal member 112 includes bore(s) 126 extending longitudinally through middle dynamic seal member 112, through which movable member(s) 40 may translate in distal and proximal directions. Middle dynamic seal member 112 may be variably adjustable with respect to the dimensions and shape of bore(s) 126 so as to advantageously increase or decrease friction on movable member(s) 40 translating through one or more bores 126 as is necessary, desirable, or preferable. Middle dynamic seal member 112 may include deflection bore(s) 130 extending longitudinally through middle dynamic seal member 112 through which movable member(s) such as deflection wires 402, 404 may translate in distal and proximal directions. Deflection bore(s) 130 are configured to align longitudinally with seal bore(s) 128 in arcuate seals 114, through which deflection wires 402, 404 may translate in distal and proximal directions. Alternatively, or additionally, though not shown in the example illustrated in FIG. 2, movable member(s) may be electrical or fiber optic wires that may pass through deflection bore(s) 130 to provide power, two-way signaling, or light at distal end 14 to power LED(s), other sensors, circuits, or light.

Bore(s) 126 may generally have a larger diameter than deflection bore(s) 130 or seal bore(s) 128. Alternatively, bore(s) 126 may generally have the same diameter as, or even a smaller diameter than, deflection bore(s) 130 or seal bore(s) 128. Deflection bore(s) 130 and seal bore(s) 128 may have approximately the same diameter and general size.

Each of seals 110 and 116 and arcuate seals 114 may be formed of flexible silicone, rubber, plastic, elastomer, amorphous sealing material, or any other material suitable to substantially prevent (or at least impede) fluid from flowing across and/or permeating through it. Each of upper dynamic seal member 106, middle dynamic seal member 112, or lower dynamic seal member 118 may be formed of nitrile (Buna), neoprene, ethylene propylene diene monomer (EPDM) rubber, silicone, fluorocarbon (Viton), polytetrafluoroethylene (PTFE, Teflon), perfluoroelastomers (FFKM), or any elastomer. Additionally, each of upper dynamic seal member 106, middle dynamic seal member 112, or lower dynamic seal member 118 may be encapsulated by an additional material such as fluorinated ethylene propylene (FEP) copolymer.

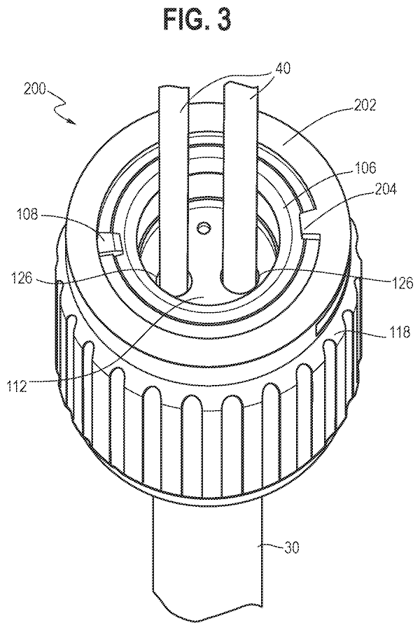

FIG. 3 illustrates a perspective view of an example of a dynamic seal 200 as assembled. Proximal to lower dynamic seal member 118 is catch 202. Stop 108 of upper dynamic seal member 106 may confront protrusion 204 on catch 202, to preclude complete 360-degree rotation of upper dynamic seal member 106 and lower dynamic seal member 118 about the longitudinal axis relative to middle dynamic seal members, accessory channels, seals, and deflection wires. Dynamic seal 200 includes a seal or a plurality of seals configured to prevent fluid, including pressurized fluid, from flowing proximally to upper dynamic seal member 106 while movable member(s) 40 may translate in proximal or distal directions. In examples of the present disclosure, movable member(s) 40 may include a single movable member or a plurality of movable members, as is illustrated in FIG. 3, which illustrates two movable members 40. In certain examples, dynamic seal may include a seal deployed to prevent fluid from flowing proximally to upper dynamic seal member 106 while a single movable member, or, alternatively, a plurality of movable members, may translate in proximal or distal directions. In other examples, a dynamic seal may include a plurality of seals deployed to prevent fluid from flowing proximally to upper dynamic seal member 106 while a single movable member, or, alternatively, a plurality of movable members, may translate in proximal or distal directions.

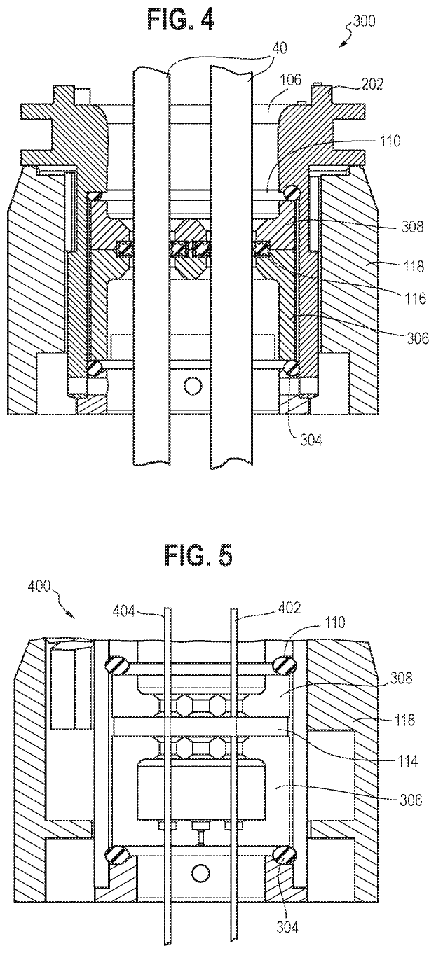

FIG. 4 illustrates a diametrical longitudinal cross-sectional view of an example of a dynamic seal 300 as assembled, with the diametrical longitudinal cross-section through the diameter of proximal middle dynamic seal member 308 and distal middle dynamic seal member 306 that includes the diameters of bores 126 and the diameters of movable members 40. As illustrated by FIG. 4, dynamic seal 300 includes upper dynamic seal member 106, which is positioned inside catch 202 and through which movable members 40 extend longitudinally. Seal 110 is seated longitudinally between upper dynamic seal member 106 and proximal middle dynamic seal member 308. Encircling each movable member 40 is a seal 116, which is seated within complementing recesses in the distal surface of proximal middle dynamic seal member 308 and complementing recesses in the proximal surface of distal middle dynamic seal member 306. Seal 304 is seated longitudinally between distal middle dynamic seal member 306 and the proximal end of elongate tube 30. Lower dynamic seal member 118 encircles proximal middle dynamic seal member 308, distal middle dynamic seal member 306, seals 110, 116, 304, and movable members 40, and lower dynamic seal member 118 and upper dynamic seal member 106 may be rotationally decoupled such that proximal middle dynamic seal member 308, distal middle dynamic seal member 306, seals 110, 116, 304, and movable members 40 remain stationary when lower dynamic seal member 118 and upper dynamic seal member 106 are rotated. Seals 110, 116, and 304 are positioned, and thereby configured, to prevent fluid, including pressurized fluid, from flowing proximally to upper dynamic seal member 106, while movable members 40 may translate proximally and distally.

FIG. 5 illustrates a chordal longitudinal cross-sectional view of an example of a dynamic seal 400 as assembled, with the chordal longitudinal cross-section parallel to the cross-section taken to illustrate FIG. 4, and through arcuate seal 114. Deflection wires 402, 404 are movable members passing through proximal middle dynamic seal member 308, arcuate seal 114, and distal middle dynamic seal member 306, with arcuate seal 114 seated in complementary recesses in distal surface of proximal middle dynamic seal member 308 and proximal surface of distal middle dynamic seal member 306. Arcuate seal 114 is positioned, and thereby configured, to prevent fluid, including pressurized fluid, from flowing proximally to upper dynamic seal member 106, while deflection wires 402, 404 may translate proximally and distally.

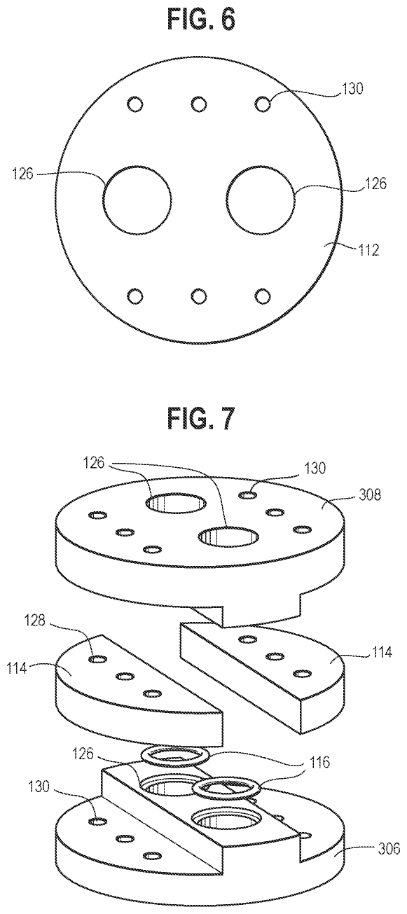

FIG. 6 illustrates a top view of an example of middle dynamic seal member 112. Middle dynamic seal member 112 includes a plurality of bores 126 extending longitudinally through middle dynamic seal member 112, and each of which through movable members 40 that are accessory channels may translate proximally or distally. Middle dynamic seal member 112 includes a plurality of deflection bores 130 extending longitudinally through middle dynamic seal member 112, and each of which through a movable member that is a deflection wire, such as deflection wire 402 or deflection wire 404, may translate proximally or distally.

FIG. 7 illustrates a perspective view of an example of proximal middle dynamic seal member 308, distal middle dynamic seal member 306, arcuate seals 114, and seals 116 as they may be disposed for assembly in an example of a dynamic seal according to the principles of the present disclosure. Proximal middle dynamic seal member 308 and distal middle dynamic seal member 306 include a plurality of bores 126 extending longitudinally and coaxially through proximal middle dynamic seal member 308 distal middle dynamic seal member 306 such that when the elements illustrated in FIG. 7 are assembled in a dynamic seal, each of the plurality of bores 126 in proximal middle dynamic seal member 308 lines up with a corresponding bore 126 in distal middle dynamic seal member 306 such that a movable member 40 such as an accessory channel may translate proximally and distally through a bore 126 in proximal middle dynamic seal member 308 and a corresponding bore 126 in distal middle dynamic seal member 306. Proximal middle dynamic seal member 308 and distal middle dynamic seal member 306 also each includes a plurality of deflection bores extending longitudinally through proximal middle dynamic seal member 308 and distal middle dynamic seal member 306 such that when the elements illustrated in FIG. 7 are assembled in a dynamic seal, each of the plurality of deflection bores 130 in proximal middle dynamic seal member 308 lines up with a corresponding deflection bore 130 in distal middle dynamic seal member 306 such that a movable member such as a deflection wire 402 or 404 may translate proximally and distally through a deflection bore 130 in proximal middle dynamic seal member 308 and a corresponding deflection bore 130 in distal middle dynamic seal member 306. Each arcuate seal 114 includes a plurality of seal bores 128, and arcuate seals 114 may be seated and compressed between recesses in the distal surface of proximal middle dynamic seal member 308 and the proximal surface of distal middle dynamic seal member 306 such that when the elements illustrated in FIG. 7 are assembled in a dynamic seal, each of the plurality of deflection bores 130 in proximal middle dynamic seal member 308 lines up with a corresponding seal bore 128 in an arcuate seal 114 and with a corresponding deflection bore 130 in distal middle dynamic seal member 306 such that a movable member such as a deflection wire 402 or 404 may translate proximally and distally through each of a plurality of deflection bores 130 in proximal middle dynamic seal member 308, a corresponding seal bore 128 in arcuate seal 114, and a corresponding deflection bore 130 in distal middle dynamic seal member 306. Seals 116 are flexible and seated in each bore 126 in slight recesses in the distal surface of proximal middle dynamic seal member 308 and the proximal surface of distal middle dynamic seal member 306 such that seals 116 are compressed between proximal middle dynamic seal member 308 and distal middle dynamic seal member 306 when the elements illustrated in FIG. 7 are assembled in a dynamic seal.

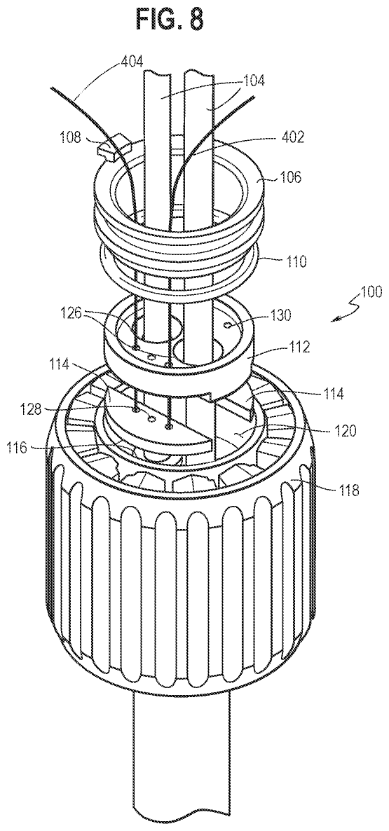

FIG. 8 illustrates the example of the elements of the dynamic seal 100 of FIG. 2 including deflection wires 402 and 404, each of which pass through upper dynamic seal member 106, seal 110, deflection bores 130 in middle dynamic seal member 112, and seal bores 130 in arcuate seals 114. Dynamic seal 100 is configured such that deflection wires 402 and 404 may translate distally and proximally while fluid, including pressurized fluid, does not flow proximally to upper dynamic seal member 106.

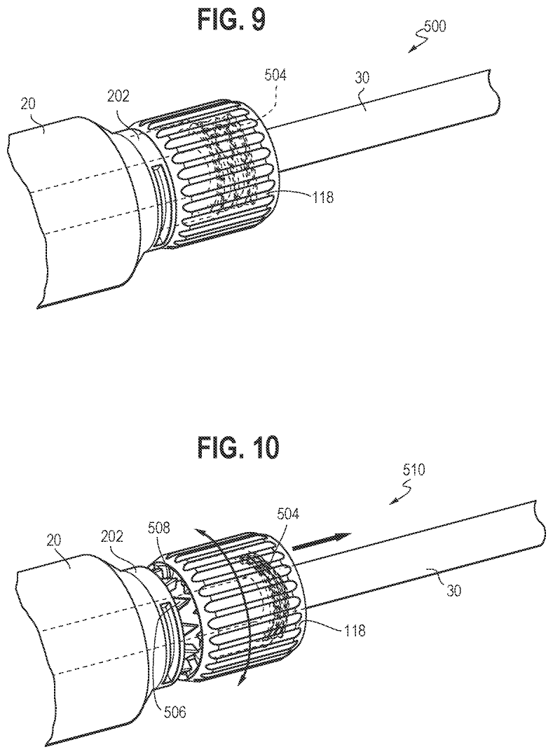

FIG. 9 illustrates a perspective view of an example of a handle 20 including an example of a dynamic seal 500 with a lower dynamic seal member 118 shown as see-through or transparent for purposes of illustration to show a spring 504 within lower dynamic seal member 118. Lower dynamic seal member 118 may translate distally relative to catch 202 and handle 20, but is biased to the locked configuration in which lower dynamic seal member 118 does not translate distally. In the locked configuration, elongate tube 30 is not rotatable about the longitudinal axis relative to handle 20. Spring 504 is positioned about the circumference internal to lower dynamic seal member 118, and biases lower dynamic seal member 118 into the locked configuration with proximal end of lower dynamic seal member 118 fronting distal end of catch 202.

FIG. 10 illustrates a perspective view of the example of the handle 20 of FIG. 9 with the lower dynamic seal member 118 shown as see-through or transparent, the lower dynamic seal member 118 moved distally away from catch 202 to compress spring 504 internal to lower dynamic seal member 118. When lower dynamic seal member 118 is translated distally, lower dynamic seal member 118 is in an unlocked configuration, in which a plurality of dynamic seal member teeth 508 that may be disposed about the proximal inner surface of lower dynamic seal member 118 are disengaged from a plurality of catch teeth 506, and spring 504 is compressed. When lower dynamic seal member 118 is in an unlocked configuration, an operator may rotate lower dynamic seal member 118 circumferentially by applying torque, thereby rotating elongate tube 30 relative to catch 202. Plurality of catch teeth 506 may be disposed about distal circumference of catch 202. When the operator releases lower dynamic seal member 118, spring 504 is released, thereby moving lower dynamic seal member 118 proximally back into the locked configuration as illustrated in FIG. 9. Plurality of dynamic seal member teeth 508 have a mating geometry with plurality of catch teeth 506 such that in the locked configuration, plurality of dynamic seal member teeth 508 mesh with plurality of catch teeth 506 so as to prevent rotation of elongate tube 30 about the longitudinal axis relative to handle 20.

A handle with a locking mechanism as described herein may be used in a number of medical device systems, particularly endoscopes. In particle, the handle of the present disclosure may be used in conjunction with the scopes, devices, and systems described in U.S. patent application Ser. No. 15/445,318 and U.S. patent application Ser. No. 15/445,518, the entire contents of both of which are hereby incorporated by reference herein in their entireties.

Although the present disclosure has been described with reference to examples and the accompanying drawings, the present disclosure is not limited thereto, but may be variously modified and altered by those skilled in the art to which the present disclosure pertains without departing from the spirit and scope of the present disclosure.

The subject-matter of the disclosure may also relate, among others, to the following aspects:

A first aspect relates to a medical device, comprising: an elongate tube comprising a lumen extending therethrough, the elongate tube defining a longitudinal axis therethrough; a movable member extending longitudinally at least partially within the lumen; and a dynamic seal, comprising: a dynamic seal member at a proximal end of the elongate tube, the movable member configured to translate proximally and distally through the dynamic seal member; and a flexible seal through which the movable member extends longitudinally; and wherein the dynamic seal member is configured to prevent fluid from flowing proximally to the dynamic seal member.

A second aspect relates to the medical device of aspect 1, wherein the flexible seal is an elastomeric seal.

A third aspect relates to the medical device of any preceding aspect, wherein the flexible seal comprises an amorphous sealing material.

A fourth aspect relates to the medical device of any preceding aspect, wherein the movable member is an accessory channel of an endoscope system.

A fifth aspect relates to the medical device of any one of aspects 1 to 3, wherein the movable member is a deflection wire of an endoscope system.

A sixth aspect relates to the medical device of any one of aspects 1 to 3, wherein the movable member is an electrical wire or a fiber-optic wire of an endoscope system.

A seventh aspect relates to the medical device of any preceding aspect, comprising a plurality of movable members, wherein the dynamic seal comprises a plurality of flexible seals; and wherein one movable member of the plurality of movable members each extends through one flexible seal of the plurality of flexible seals.

An eighth aspect relates to the medical device of any preceding aspect, further comprising a second dynamic seal member; wherein the dynamic seal member is within the second dynamic seal member; and wherein the second dynamic seal member is rotationally decoupled from the dynamic seal member.

A ninth aspect relates to the medical device of any preceding aspect, wherein the dynamic seal member is variably adjustable and configured to increase or decrease friction on the movable member.

A tenth aspect relates to a medical device, comprising: an elongate tube comprising a lumen extending therethrough, the elongate tube defining a longitudinal axis therethrough; a movable member extending longitudinally at least partially within the lumen; a first dynamic seal member at a proximal end of the elongate tube, the movable member configured to translate proximally and distally through the first dynamic seal member; a flexible seal through which the movable member extends longitudinally; and a second dynamic seal member rotationally decoupled from the first dynamic seal member; wherein the first dynamic seal member is within the second dynamic seal member; and wherein the first dynamic seal member is configured to prevent fluid from flowing proximally to the first dynamic seal member.

An eleventh aspect relates to the medical device of aspect 10, wherein the flexible seal is an elastomeric seal.

A twelfth aspect relates to the medical device of any one of aspects 10 or 11 wherein the flexible seal comprises an amorphous sealing material.

A thirteenth aspect relates to the medical device of any one of aspects 10 to 12, wherein the movable member is an accessory channel of an endoscope system.

A fourteenth aspect relates to the medical device of any one of aspects 10 to 12, wherein the movable member is a deflection wire of an endoscope system.

A fifteenth aspect relates to the medical device of any one of aspects 10 to 12, wherein the movable member is an electrical wire or a fiber-optic wire of an endoscope system.

A sixteenth aspect relates to the medical device of any one of aspects 10 to 15, comprising a plurality of movable members; wherein the first dynamic seal member comprises a plurality of flexible seals; and wherein one movable member of the plurality of movable members each extends through one flexible seal of the plurality of flexible seals.

A seventeenth aspect relates to the medical device of any one of aspects 10 to 16, further comprising a catch proximal to the second dynamic seal member; and wherein the second dynamic seal member is configured to move between a locked configuration and an unlocked configuration, the second dynamic seal member being biased to the locked configuration.

An eighteenth aspect relates to the medical device of aspect 17, wherein, when the second dynamic seal member is in the unlocked configuration, the second dynamic seal member is rotatable about the longitudinal axis relative to the first dynamic seal member.

A nineteenth aspect relates to the medical device of aspect 17, wherein, when the second dynamic seal member is in the locked configuration, the second dynamic seal member engages with the catch and is not rotatable about the longitudinal axis relative to the first dynamic seal member.

A twentieth aspect relates to a medical device, comprising: an elongate tube comprising a lumen extending therethrough, the elongate tube defining a longitudinal axis therethrough; a movable member extending longitudinally at least partially within the lumen; and a dynamic seal, comprising: a dynamic seal member at a distal end of the elongate tube, the movable member configured to translate proximally and distally through the dynamic seal member; and a flexible seal through which the movable member extends longitudinally; and wherein the dynamic seal member is configured to prevent fluid from flowing proximally to the dynamic seal member.

In addition to the features mentioned in each of the independent aspects enumerated above, some examples may show, alone or in combination, the optional features mentioned in the dependent aspects and/or as disclosed in the description above and shown in the figures.

What is claimed is:

1. A medical device, comprising:
an elongate tube comprising a lumen extending therethrough, the elongate tube defining a longitudinal axis therethrough;
a movable member extending longitudinally at least partially within the lumen; and
a dynamic seal, comprising:
a dynamic seal member at a proximal end of the elongate tube, the movable member configured to translate proximally and distally through the dynamic seal member; and
a flexible seal through which the movable member extends longitudinally; and
wherein the dynamic seal member is configured to prevent fluid from flowing proximally to the dynamic seal member.

2. The medical device of claim 1, wherein the flexible seal is an elastomeric seal.

3. The medical device of claim 1, wherein the flexible seal comprises an amorphous sealing material.

4. The medical device of claim 1, wherein the movable member is an accessory channel of an endoscope system.

5. The medical device of claim 1, wherein the movable member is a deflection wire of an endoscope system.

6. The medical device of claim 1, wherein the movable member is an electrical wire or a fiber-optic wire of an endoscope system.

7. The medical device of claim 1, comprising a plurality of movable members;
wherein the dynamic seal comprises a plurality of flexible seals; and
wherein one movable member of the plurality of movable members each extends through one flexible seal of the plurality of flexible seals.

8. The medical device of claim 1, further comprising a second dynamic seal member;
wherein the dynamic seal member is within the second dynamic seal member; and
wherein the second dynamic seal member is rotationally decoupled from the dynamic seal member.

9. The medical device of claim 1, wherein the dynamic seal member is variably adjustable and configured to increase or decrease friction on the movable member.

10. A medical device, comprising:
an elongate tube comprising a lumen extending therethrough, the elongate tube defining a longitudinal axis therethrough;
a movable member extending longitudinally at least partially within the lumen;
a first dynamic seal member at a proximal end of the elongate tube, the movable member configured to translate proximally and distally through the first dynamic seal member;
a flexible seal through which the movable member extends longitudinally; and
a second dynamic seal member rotationally decoupled from the first dynamic seal member;
wherein the first dynamic seal member is within the second dynamic seal member; and
wherein the first dynamic seal member is configured to prevent fluid from flowing proximally to the first dynamic seal member.

11. The medical device of claim 10, wherein the flexible seal is an elastomeric seal.

12. The medical device of claim 10, wherein the flexible seal comprises an amorphous sealing material.

13. The medical device of claim 10, wherein the movable member is an accessory channel of an endoscope system.

14. The medical device of claim 10, wherein the movable member is a deflection wire of an endoscope system.

15. The medical device of claim 10, wherein the movable member is an electrical wire or a fiber-optic wire of an endoscope system.

16. The medical device of claim 10, comprising a plurality of movable members;
wherein the first dynamic seal member comprises a plurality of flexible seals; and
wherein one movable member of the plurality of movable members each extends through one flexible seal of the plurality of flexible seals.

17. The medical device of claim 10, further comprising a catch proximal to the second dynamic seal member; and
wherein the second dynamic seal member is configured to move between a locked configuration and an unlocked configuration, the second dynamic seal member being biased to the locked configuration.

18. The medical device of claim 17, wherein, when the second dynamic seal member is in the unlocked configuration, the second dynamic seal member is rotatable about the longitudinal axis relative to the first dynamic seal member.

19. The medical device of claim 17, wherein, when the second dynamic seal member is in the locked configuration, the second dynamic seal member engages with the catch and is not rotatable about the longitudinal axis relative to the first dynamic seal member.

20. A medical device, comprising:
an elongate tube comprising a lumen extending therethrough, the elongate tube defining a longitudinal axis therethrough;
a movable member extending longitudinally at least partially within the lumen; and
a dynamic seal, comprising:
a dynamic seal member at a distal end of the elongate tube, the movable member configured to translate proximally and distally through the dynamic seal member; and
a flexible seal through which the movable member extends longitudinally; and
wherein the dynamic seal member is configured to prevent fluid from flowing proximally to the dynamic seal member.
